(19) United States
(12) Patent Application Publication
(10) Pub. No.: US 2003/0109887 A1
(43) Pub. Date: Jun. 12, 2003

(54) Medical graft component and methods of installing same

(75) Inventors: Jason A. Galdonik, St. Louis Park, MN (US); William J. Swanson, St. Paul, MN (US); Mark D. Wahlberg, St. Paul, MN (US); Todd Allen Berg, Stillwater, MN (US)

Correspondence Address: FISH & NEAVE, 1251 AVENUE OF THE AMERICAS, 50TH FLOOR, NEW YORK, NY, 10020-1105, US

(73) Assignee: St. Jude Medical ATG, Inc.

(21) Appl. No.: 10/345,470

(22) Filed: Jan. 15, 2003

Related U.S. Application Data
(63) Continuation of application No. 09/187,361, filed on Nov. 6, 1998.

(51) Int. Cl. A61F 11/00; A61B 17/08

(52) U.S. Cl. 606/108; 606/153

(57) ABSTRACT

Apparatus for securing an axial end portion of a tubular graft conduit in a lumen of a patient's existing tubular body organ structure via an aperture in a side wall thereof is disclosed. An anchor device is configured for attachment to the end portion of the tubular graft conduit. The anchor device defines a constant axial length and a cross-section radially expandable between a first diameter sized for insertion into the aperture in the side wall of the body conduit and a second diameter sized to secure the end portion of the tubular graft conduit coaxially between the anchor device and the lumen of the tubular body conduit. The anchor device defines a longitudinal axis that may change between a substantially straight configuration and a curvilinear configuration such that the cross-section remains substantially unchanged.

This application is a continuation of U.S. patent application Ser. No. 09/187,361, filed Nov. 6, 1998, which is hereby incorporated by reference herein in its entirety.

### BACKGROUND OF THE INVENTION

This invention relates to structures that can be used to make connections between tubular medical grafts and a patient's tubular body conduits. The invention also relates to methods for making and using the structures mentioned above.

Tubular grafts are frequently needed in medical procedures. For example, a coronary bypass procedure may involve the installation of a tubular graft between an aperture that has been formed in the side wall of the aorta and an aperture that has been formed in the side wall of a coronary artery downstream from an occlusion or blockage in that artery. Each end of the graft must be connected to either the aorta or the coronary artery. Each such connection must extend annularly around the associated end of the graft conduit and be fluid-tight so that no blood will leak out. One common way to produce such connections is by suturing. It will be appreciated, however, that making such connections by suturing can be extremely difficult, time-consuming, and dependent on the skill of the physician for the quality of the results. There is also increasing interest in less invasive procedures which tend to impose constraints on the physician's access to the sites at which graft connections must be made and thereby make it more difficult or even impossible to use suturing to make such connections (see, for example, Goldsteen et al. U.S. Pat. No. 5,976,178, Sullivan et al. U.S. Pat. No. 6,120,432, Sullivan et al. U.S. patent application Ser. No. 08/869,808, filed Jun. 5, 1997, Berg et al. U.S. Pat. No. 6,475,222, and Peterson et al. U.S. Pat. No. 6,152,937, all of which are hereby incorporated by reference herein in their entireties).

A conventional suturing technique is illustrated at FIG. 1. Sutures 100 are typically applied to a proximal anastomosis site 102, i.e., at the joining of a graft conduit 104 with the side wall of the aorta 106 and a distal anastomosis site 108, i.e., at the joining of the graft conduit 104 with the side wall of the coronary artery 110, typically downstream of the blockage 112. Failure of the bypass circuit often occurs at the distal anastomosis site 108 due to injury or to poor fluid dynamics. Such tissue stress may trigger a healing response that ultimately reduces the patency of the graft.

Typical causes of this failure at the distal anastomosis site 108 may include a poor flow transition from the direction of flow in the graft 104 (arrow A) to the direction of flow in the coronary artery 110 (arrow B). This abrupt transition in flow direction is less than optimal, and often results in turbulent flow and “jetting,” which may injure the blood vessels in the area. The poor flow transition may also create a competitive flow condition. Blood entering the coronary artery 110 from the graft 104 may initially flow both upstream and downstream. The upstream flow competes with the native downstream flow in the coronary artery. Consequently, a slow flow condition may result, which is known to produce thrombus.

In addition to poor flow dynamics, conventional suturing techniques may contribute to the failure of the distal anastomosis. The sutures 100 themselves may initiate injury to the graft vessel at coronary anastomosis site, which is already in high stress. When veins, such as the saphenous vein, are used for graft material, the high arterial pressure may dilate the vein to a larger diameter than it would experience under typical venous pressure. At the anastomosis site, the combination of the sutures and the arterial pressure amplifies the stress on the tissue, resulting in tissue injury and reduced patency.

In view of the foregoing, it is an object of this invention to provide improved and simplified apparatus and methods for connecting two tubular structures while minimizing stress to the tissue being joined.

It is still another object of this invention to provide improved and simplified methods of making structures that can be used as medical graft anchor apparatus.

It is yet another object of this invention to provide improved and simplified methods for installing medical graft anchor apparatus.

### SUMMARY OF THE INVENTION

This and other objects of the invention are accomplished in accordance with the principles of the invention by providing methods and apparatus for securing an axial end portion of a tubular graft conduit in a lumen of a patient's existing tubular body organ structure via an aperture in a side wall thereof. In accordance with the invention, an anchor device is configured for attachment to the end portion of the tubular graft conduit. The anchor device defines a constant axial length and a cross-section radially expandable between a first diameter sized for insertion into the aperture in the side wall of the existing tubular body organ structure and a second diameter sized to secure the end portion of the tubular graft conduit coaxially between the anchor device and the lumen of the tubular body conduit.

In a preferred embodiment, the anchor device defines a longitudinal axis that is movable between a substantially straight configuration and a curvilinear configuration while maintaining a constant cross-sectional area to conform to the existing body structure. The anchor structure includes a plurality of axial members that are relatively radially movable to define the first and second diameters. In addition, the axial members may be provided with expansion links which allow each axial member to independently elongate to conform to the desired curvature.

Further features of the invention, its nature and various advantages will be more apparent from the accompanying drawings and the following detailed description of the preferred embodiments.

### BRIEF DESCRIPTION OF THE DRAWINGS

FIG. 1 is a simplified schematic view of the prior art anastomosis technique.

FIG. 2 is a perspective view of an illustrative embodiment of a component according to this invention.

FIG. 3 is a perspective view of a portion of the component of FIG. 2 in a second configuration.

FIG. 4 is a perspective view of a portion of the component of FIG. 2 in a third configuration.

FIG. 5 is a simplified perspective view of the FIG. 2 component, a graft conduit, and additional apparatus for mounting the component to the graft conduit according to the invention.

FIG. 6(a) is a simplified perspective view, illustrating the component of FIG. 2 mounted to the graft conduit, thereby forming a graft assembly.

FIG. 6(b) is a simplified perspective view, similar to FIG. 6(a), illustrating the component of FIG. 2 mounted to the graft conduit according to an alternative embodiment.

FIG. 7 is a sectional view of the FIG. 6 assembly, and additional apparatus for delivering the graft assembly in a tubular body conduit in a first configuration.

FIG. 8 is a sectional view similar to FIG. 7, illustrating the delivery apparatus in a second configuration.

FIG. 9 is a simplified longitudinal view showing a portion of an illustrative procedure and related apparatus in accordance with this invention.

FIG. 10 is a simplified longitudinal view showing additional apparatus according to an alternative embodiment of the subject invention.

FIG. 11(a) is partial simplified longitudinal section similar to FIG. 9, showing alternative apparatus and a stage in an illustrative procedure.

FIG. 11(b) is partial simplified longitudinal section similar to FIG. 11(a), showing a later stage in the illustrative procedure of FIG. 11(a).

FIG. 11(c) is partial simplified longitudinal section similar to FIG. 11(b), showing an even later stage in the illustrative procedure of FIG. 11(a).

FIG. 12 is a simplified longitudinal sectional view showing a still later stage in the illustrative procedure depicted in part by FIGS. 11(a)-11(c) and related apparatus shown in FIGS. 7-8.

FIG. 13 is an enlarged longitudinal sectional view showing an even later stage in the illustrative procedure depicted in part by FIG. 12.

FIG. 14 is a simplified longitudinal sectional view of the assembly shown in FIG. 6 installed in the tubular body conduit.

FIG. 15 is a simplified longitudinal sectional view similar to FIG. 12, illustrating alternative procedure and apparatus according to the invention.

FIG. 16 is a simplified longitudinal sectional view similar to FIG. 12, illustrating another alternative procedure and apparatus according to the invention.

FIG. 17 is a simplified longitudinal sectional view similar to FIG. 12, illustrating still another alternative procedure and apparatus according to the invention.

### DETAILED DESCRIPTION OF THE PREFERRED EMBODIMENTS

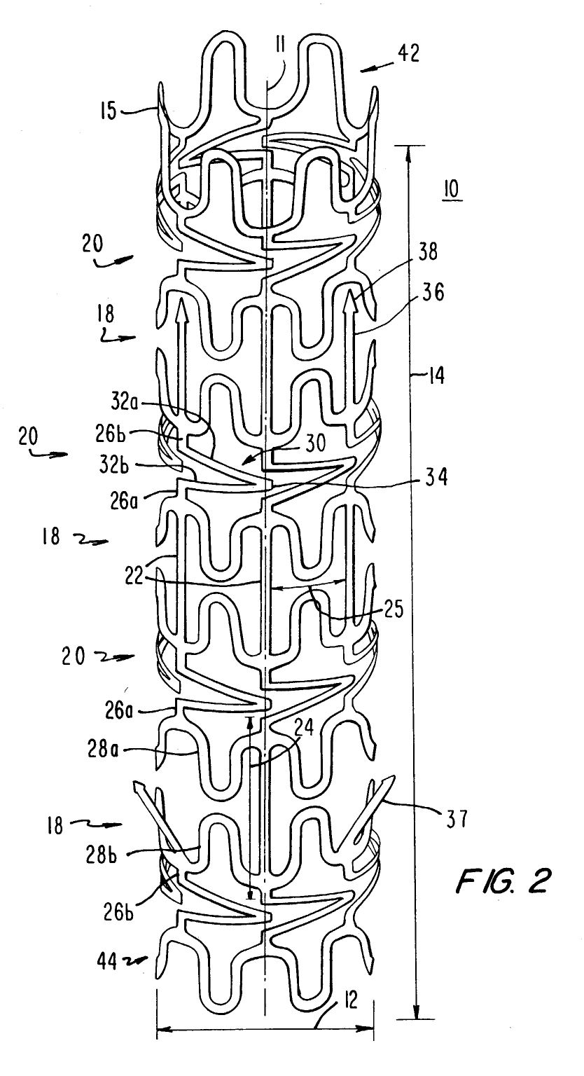

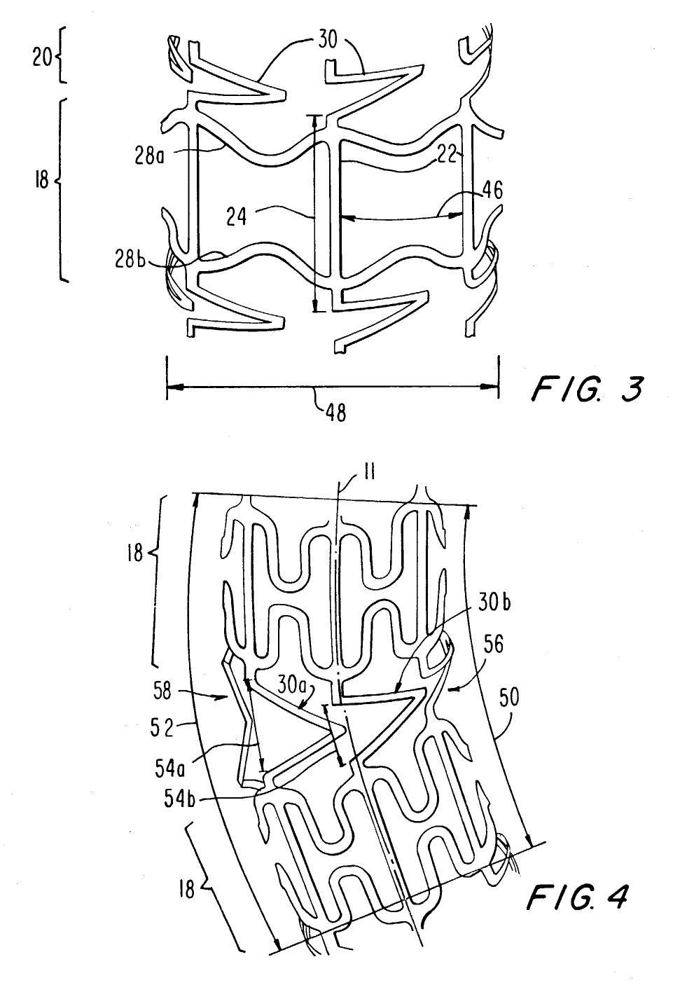

FIG. 2 illustrates a preferred embodiment of a component 10 for use in anchoring an end portion of a tubular graft conduit within the lumen of a patient's tubular body conduit. In order to facilitate the intraluminal placement and attachment of component 10, it is manufactured in a substantially toroidal configuration, defining an axis 11 and a substantially curved cross-section. Axis 11 is depicted as a straight line in FIG. 2, although it is contemplated that axis 11 may be curvilinear to conform to a tubular body conduit (see, FIG. 4). Moreover, cross-section may be circular, elliptical or any other substantially closed curve to likewise conform to the lumen of the tubular body conduit. In a preferred embodiment, component 10 is substantially cylindrical and defines an initially linear axis 11, a circular cross-section 15 having an initial diameter 12, and an initial length 14. Component 10 may be used to secure a graft conduit within a coronary artery, and therefore, the initial diameter may be approximately 1 mm and the initial length may be approximately 1.5 mm. Component 10 may be changed from an initial configuration (FIG. 2), to an expanded configuration illustrated in FIG. 3. As will be described in greater detail below, component 10 may have a radially expanded diameter 48, while maintaining a constant cross-sectional configuration and a substantially constant overall length. Component 10 may also be curved to conform to the curvature of tubular body conduits, as illustrated in FIG. 4. During such curvature, axis 11 assumes a curvilinear configuration while the cross-sectional configuration remains substantially unchanged. Therefore, component 10 experiences no distortion of its geometry, as will also be described below.

Component 10 may be fabricated from material having resilient or plastically deformable characteristics to permit the component to assume at least the initial configuration (FIG. 2), the expanded configuration (FIG. 3), and the curved configuration (FIG. 4). For example, component 10 may be fabricated from stainless steel. In the above example, the thickness of the sheet may be selected as 0.004 inches. Alternatively, component 10 may be fabricated from other metals, such as tantalum to improve radiopacity. Self-expanding materials, such as nickel-titanium may be used, as will be described in greater detail below with respect to the installation of component 10.

In order to fabricate component 10 into the shape illustrated in FIG. 2, a preferred method of construction begins with a cylindrical tube (not shown) of a material such as one of those described above. The length and diameter of the cylindrical tube corresponds to the initial diameter 12 and length 14 of the component 10 in its initial configuration. The uncut sheet may be cut or machined, preferably laser cut and ground, into the configuration illustrated in FIG. 2. Alternatively, component 10 may be constructed from a sheet of a material, such as that described above, having a length corresponding to the initial length 14 and a width corresponding to the circumference of component 10. The sheet is subsequently cut and formed into the configuration illustrated in FIG. 2.

The configuration of component 10 includes a series of annular cells that may be repeated as many times as necessary to achieve the length of component required. An expansion cell 18 permits component 10 to vary in diameter without substantial change to cross-sectional shape, e.g., circular, or to axial length. An articulation cell 20 is provided in order to allow component 10 to bend or curve without distortion of its geometry. The dimensions of the cells may be selected in order to conform to the flexibility and thickness of the tubular body conduit. For example, if the vessel is capable of assuming small radius curves, it may be necessary to space the articulation cells closer together in order to conform to the vessel. Alternatively, articulation cells may be more widely spaced if the tubular body conduit is more rigid. Expansion cells 18 and articulation cells 20 are configured to operate independently of one another. For example, one portion of the tubular body conduit may have a diameter larger or smaller than the other portion, which would require differential expansion of expansion cells 18. In addition, the radius curvature of the tubular conduit may vary along the length thereof, and thus require differential displacement of the various articulation cells 20.

Expansion cells 18 include a series of axially aligned struts 22 having an axial length 24. Struts 22 are uniformly spaced about the circumference of the cell, by distance 25. A plurality of circumferential expansion members 28a and 28b are connected to axially aligned struts 22. More particularly, adjacent an axial end 26a of each strut 22 is a first expansion member 28a, and adjacent axial end 26b is a second expansion member 28b. In the initial configuration of FIG. 2, expansion members 28a and 28b have a compact serpentine shape. Alternatively, expansion members 28a and 28b may have a zig-zag shape.

Articulation cells 20 are typically positioned between adjacent expansion cells 18, although it is contemplated that a plurality of articulation cells 20 may be positioned consecutively. Each articulation cell 20 includes a plurality of expansion links 30, each of which operates independently of the adjacent connections. A preferred embodiment of the expansion link 30 has a “V” configuration, with a first arm 32a connected to axial strut end 26b and a second arm 32b connected to axial strut end 26a. First and second arms 32a and 32b are interconnected at a point 34. It is contemplated that links 30 may have alternative shapes, e.g., “U”-shaped or “Σ”-shaped. Moreover, it is contemplated that expansion links may also include any member along the axial strut which permits axial elongation of the axial strut. Thus, it is contemplated that the expansion links may include resilient coils, pivot linkages, or the like.

Attachment of component 10 to a graft conduit, such as graft conduit 104, may be made by engagement members, such as members 36. In one of the plurality of expansion cells 18, at least one of the struts 22 may be modified into engagement member 36, which is connected to the cell structure at one end only. The free end 38 of member 36 is configured as a sharpened, piercing member, having barbs to prevent removal from the tissue engaged thereby. Engagement member 36 may be deflected radially outwardly to pierce the graft tissue. Alternatively, interconnection point 34 of expansion links 30 may be deflected radially outward to serve as tie-downs for sutures.

In a preferred embodiment, positioning members 37 are provided to assist in the placement of component 10 within the tubular body conduit. In a preferred embodiment, positioning members 37 are substantially axially oriented and extend radially outward to define an angle with the longitudinal axis 11. Positioning members 37 are located at a predetermined axial location on component 10 and act as stops to inhibit insertion of the component 10 into tubular body conduit beyond a predetermined depth by engaging the side wall of the tubular body conduit. As will be described below, this features also provides an indication to the physician that component 10 is properly seated within the tubular body conduit to provide a secure hemodynamic seal and inhibit native flow in the coronary artery upstream of the occlusion 112.

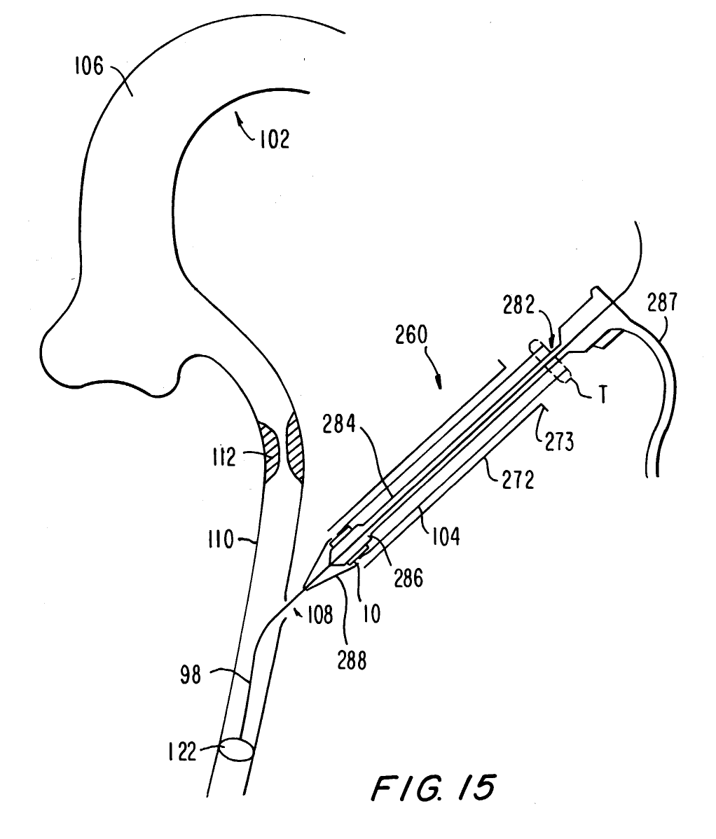

The end portions 42 and 44 are provided with a serpentine configuration which atraumatically engages the tubular members being joined, as will be described in greater detail below (See, e.g., FIG. 14).

FIG. 3 illustrates component 10 in an expanded configuration. Component 10 may be radially expanded without substantially changing the overall length of the component. This is particularly useful when implanting the component 10 in a tubular body conduit, because the end portions of the component are fixed both during and after radial expansion.

During radial expansion, each expansion cell 18 substantially maintains its overall length as well as a constant cross-sectional configuration, e.g., circular. Expansion members 28a and 28b change shape from the folded or serpentine configuration of FIG. 2 to a more nearly straightened configuration of FIG. 3. Consequently, struts 22 which were initially spaced apart distance 25 (FIG. 2) are further spaced apart to a distance 46. The overall diameter is thus expanded from the initial diameter 12 (FIG. 2), to an expanded diameter 48 while maintaining a substantially circular cross-sectional shape. In the preferred embodiment, the expanded diameter 48 is 3.5 mm. As may be seen in the FIG., the overall axial length of the cell 18 is unaffected. More particularly, length 24 of each strut 22 is substantially unchanged. (Articulation cell 20 is unaffected by the radial expansion, wherein expansion links 30 become more widely spaced without changing their “V”-shaped configuration.) In a preferred embodiment of the subject invention, the overall length 14 remains constant independent of radial expansion. It will be understood that variations in overall length may occur during radial expansion. Such variations may occur, by way of example and without limitation, due to variations in component geometry or material characteristics. However, components exhibiting such variations in overall length shall nevertheless be considered within the scope of the subject invention.

As illustrated in FIG. 4, component 10 may conform to a curved vessel without substantially altering its cross-sectional geometry. To achieve such curvature of component 10, axis 11 assumes a curvilinear shape. Typically, conforming a cylindrical member to a curved tubular vessel presents particular design problems. For example, the length of an arc 50 on the inside of a curve is substantially shorter than the length of an arc 52 on the outside of the curve. A typical prior art cylindrical member would become distorted in an attempt to conform to this shape, e.g., it may flatten at the center of the curve and thus have a reduced internal diameter. In other words, the cross-sectional configuration would not remain constant during curvature, but rather would be flattened to a narrow elliptical configuration with a reduced cross-sectional area at the point of curvature. This is problematic, especially when the component is used as a tubular graft to convey a fluid, such as a coronary artery bypass graft conveying arterial blood. The unpredictable distortion, including reduction in diameter, may seriously alter the fluid flow and introduce turbulent flow.

The component 10 according to the invention is able to conform to the curve due to the unique articulation cell structure 20. FIG. 4 illustrates component 10 conforming to the curve. Each radial expansion cell 18 is undistorted, thus the internal diameter within each cell 18 is unchanged. The expansion links 30 are able to expand independently to follow the curve. More particularly, link 30a, which is close to the outside of the curve, expands a distance 54a. Link 30b, which is located closer to the inside of the curve, expands less, i.e. distance 54b. As a result, articulation cell 20, which had a cylindrical configuration with a constant length (FIG. 2), is reconfigured to have a narrow length 56 inside the curve and a greater length 58 outside the curve. This results in minimal distortion to the shape of the cross-section of the component 10.

It is contemplated that a self-expanding component may be used to secure a graft conduit within the lumen of the body structure. A self-expanding component may be substantially similar to the configuration described with respect to FIGS. 2-3. However, the configuration shown in FIG. 3 would represent the unstressed, expanded configuration of the self-expanding component. Consequently, the configuration in FIG. 2 would represent a stressed configuration in which the component is maintained during insertion and prior to deployment wherein the self-expanding component is permitted to return to the unstressed configuration. Nevertheless, the component would maintain a constant length during radial expansion and compression.

Although the radial expansion may occur as a result of the self-expanding properties of the component, it is preferable that the articulation properties result from plastic deformations of the expansion links. More particularly, while expansion members 28a and 28b may be self-expandable, expansion links 30 are preferably plastically deformable. This enables the surgeon to exercise more control over the articulation of the component and to prevent unwanted bending of either the component or the tubular conduits being joined.

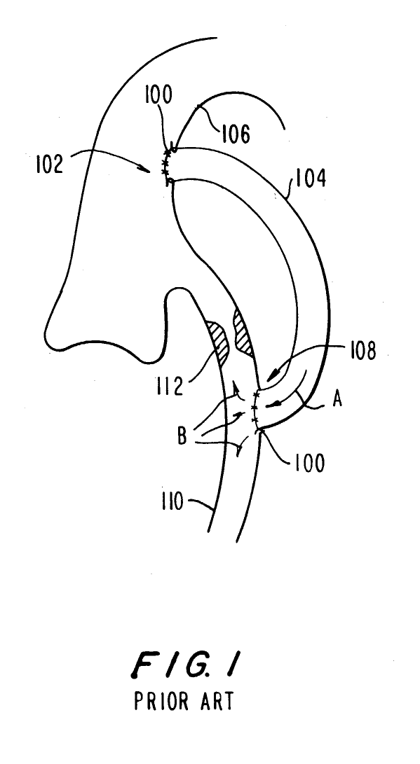

Component 10 may be used by itself as a stent. Alternatively, a tubular graft conduit, such as tubular graft conduit 104 (FIG. 1), may be connected to component 10 prior to installation in the tubular body conduit. Graft conduit 104 may be an artificial conduit or a natural body conduit, such as the saphenous vein when a coronary artery bypass graft is performed. Mandrel 61 may be used to assist in the mounting of graft conduit 104 to component 10. Mandrel 61 is configured with an atraumatic distal end portion 62 and a distal tubular portion 64 defining a first diameter 65 and a proximal tubular portion 66 defining a second diameter 67. A tapered portion 68 provides the transition between tubular portion 64 and tubular portion 66. Diameter 65 is typically marginally smaller than first diameter 12 of component 10 (See, FIG. 2), and diameter 67 is marginally smaller than the diameter of graft conduit 104. In a preferred embodiment, diameter 65 is less than 1 mm and diameter 67 is approximately 3 mm.

To attach component 10 to graft conduit 104, component 10 is initially placed coaxially over distal tubular portion 64 and graft conduit 104 is placed coaxially over proximal tubular portion 66 adjacent tapered portion 68. Expansion links 30c and 30d may be deflected radially outward to provide tie-down points for sutures 70 used to attach the graft conduit 104 thereto. The tapered portion 68 of mandrel 61 may be provided with a plurality of markers or scales 72 which provide a visual aid to the physician applying the sutures. More particularly, expansion links 30c and 30d may be aligned with scales 72 to indicate where the sutures 70 are to pass through the distal end portion 114 of the graft conduit 104. Sutures 70 are inserted through the distal end portion 114 at locations 116 and around expansion links 30c and 30d. When sutures 70 are tightened, the graft 104 is drawn distally to component 10 as indicated by the arrows C in the FIG.

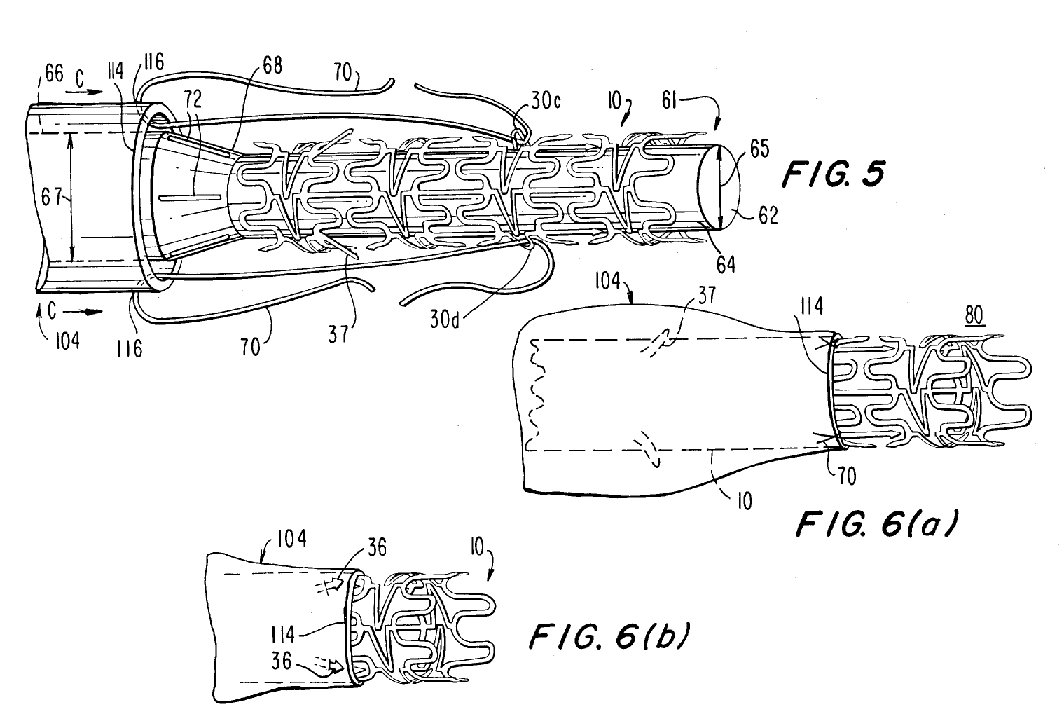

As FIG. 6(a) illustrates, further tightening of sutures 70 draws the end portion 114 of graft conduit 104 toward expansion links 30c and 30d (not shown in FIG. 6a) and securely cinches the graft conduit 104 around component 10, providing a low profile to allow insertion thereof into a small aperture in a tubular body conduit, such as the coronary artery. The cinched configuration of the end portion 114 around component 10 assists in the expansion of component 10 during deployment. When component 10 is expanded, end portion 114 may likewise expand to its original diameter without stretching or injuring the graft tissue. The resulting graft assembly 80 may include component 10 attached to graft conduit 104. When component 10 is used as a stent, i.e. without a graft conduit attached thereto, the graft assembly 80 shall refer to the component 10 only.

Alternatively, component 10 may be secured to graft conduit 104 by the engagement members 36 (FIG. 6(b)). The sharpened end portions 38 of members 36 are deflected radially outward. With a starting configuration similar to FIG. 5, the end portion 114 of graft conduit 104 is grasped by forceps and advanced over the component 10. The forceps may then be used to pierce the tissue with the engagement members 36. Since the initial diameter of component 10 is smaller than the diameter of the graft conduit 104, the end portion 114 is similarly cinched as described above with respect to FIG. 6(a).

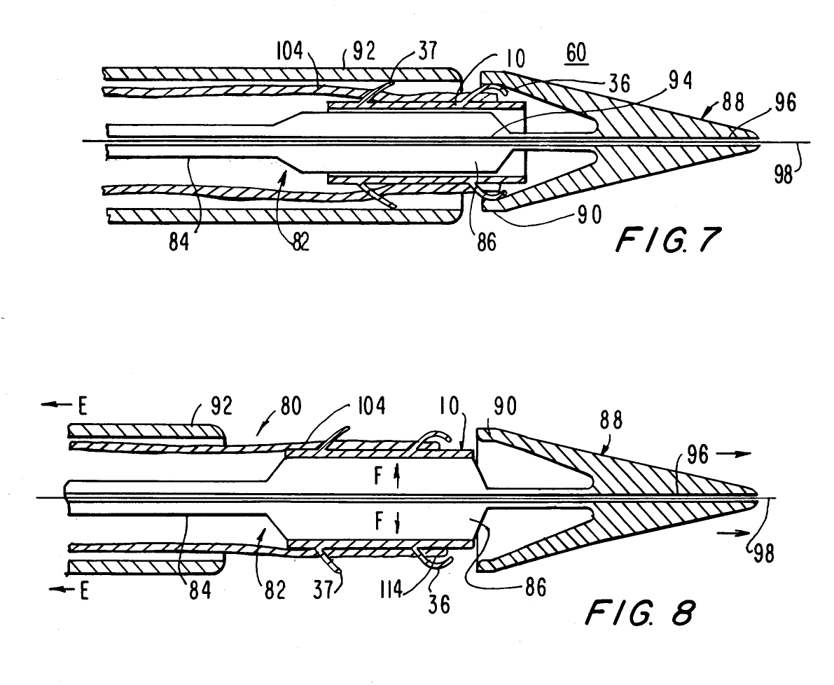

Graft assembly 80 may be installed in tubular conduits using several techniques. An exemplary apparatus 60 for delivering and deploying graft assembly 80 in the patient is illustrated in FIG. 7. Apparatus 60 may be used in a percutaneous procedure wherein connector 10 and graft conduit 104 are inserted into the patient's existing tubular body structure, e.g., the circulatory system, and deployed from inside the lumen of the body structure to the outside thereof. Alternatively, apparatus 60 may be used in minimally invasive surgical procedures, wherein an incision, access trocar or other small entry opening is provided in the patient's body. Instrument 60 should be sized to permit insertion into such opening to the operative site, and endoscopic viewing apparatus may be used to remotely view the procedure. In yet another alternative, apparatus 60 may be used in conventional surgical techniques where full access and direct visualization are appropriate.

After component 10 is attached to graft conduit 104 to form a graft assembly 80, delivery device 60 may be used to implant the graft assembly in the lumen of the tubular body conduit. Delivery device 60 may include an expandable member, such as balloon catheter 82. Balloon catheter 82 has an elongated body portion 84 with an expandable balloon structure 86. As illustrated in the FIG., balloon structure 86 is configured to have a compressed or deflated condition which is sized for insertion into component 10. An introduction cone 88 is provided with a tapered configuration to gradually dilate the aperture in the tubular body conduit without causing damage to the tissue, as will be described in greater detail below. Introduction cone 88 provides a smooth introduction surface into the tubular body conduit. In a preferred embodiment, introduction cone 88 is connected to the distal end portion of balloon catheter 82, and is movable therewith. The proximal portion 90 of cone 88 circumferentially surrounds the distal end portions of component 10 and graft conduit 104 in order to provide an atraumatic entry into the tubular body conduit. An outer sheath 92 may be provided to surround graft assembly 80 and introduction cone 88 to protect against damage during deployment. In an alternative embodiment, the sheath 92 is omitted from the delivery device. Balloon catheter 82 and introduction cone 88 are provided with lumen 94 and 96, respectively, to facilitate the delivery of the graft assembly over a longitudinal member, such as wire 98, described below.

As illustrated in FIG. 8, outer sheath 92 is retracted proximally (as indicated by arrows E) if it has been used during the procedure. In the compressed state, balloon catheter 82 is movable with respect to component 10. Balloon catheter 82 and cone 88 are both advanced distally until portion 90 of cone 88 has cleared the end portion of component 10 and does not interfere with the expansion thereof. Subsequently, body portion 84 of catheter 82 supplies a medium such as compressed air or saline to balloon structure 86 to expand the radial diameter thereof (as indicated by arrows F). The gathered/cinched configuration of graft conduit 104 (see, FIGS. 5-6) permits the end portion 114 to expand along with component 10. FIG. 8 illustrates a partial expansion of component 10. Further expansion of component 10 results in an internal diameter of component 10 which is larger than the diameter of cone 88. Consequently, cone 88 may be withdrawn proximally within and through component 10 (not shown). As will be described below in greater detail, the expansion of component 10 annularly compresses graft conduit 104 between the component 10 and the wall of the tubular body conduit to form a secure hemodynamic seal. In addition, positioning members 37 are arranged in a radial manner in order to allow a predetermined insertion of component 10 into tubular body conduit.

It is contemplated that a self-expanding component may be used to secure a graft conduit within the lumen of the body structure. Apparatus for installing the self-expanding component may be substantially similar to that shown in FIG. 7. However, the self-expanding component naturally would not require an expanding structure such as balloon structure 86. Rather, outer sheath 92 would be useful to maintain the self-expanding component in the configuration illustrated in FIG. 2. When the self-expanding component is properly positioned, sheath 92 may be withdrawn proximally in order to permit the self-expanding component to return to the deployed position of FIG. 3 and secure the graft conduit in position.

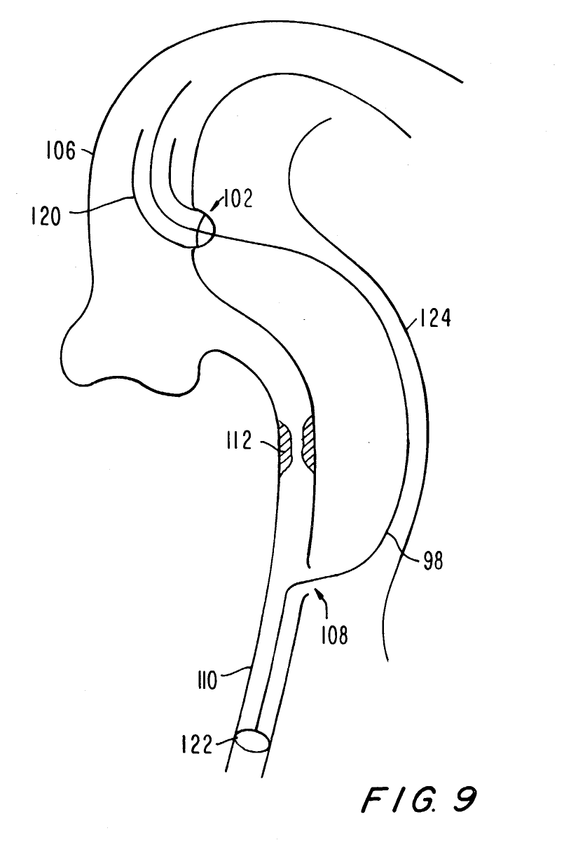

FIGS. 9-13 illustrate exemplary procedures for installing component 10 and graft conduit 104 within the lumen of a tubular body conduit. The exemplary embodiment is illustrated in connection with a coronary artery bypass graft procedure, although it is contemplated that the subject method and apparatus are applicable to other tubular graft procedures. Moreover, the procedure is illustrated in connection with a graft conduit for conveying fluid. Where component 10 is used as a stent, the procedure is substantially identical with the exception that no graft conduit is used.

FIG. 9 illustrates aorta 106 which serves as the arterial blood source for the new graft. Coronary artery 110, in this example, has an occlusion 112, which reduces the blood flow downstream to supply the heart tissue. The proximal location of the graft anastomosis to the aorta 106 is location 102. Distal location 108 is selected by the physician as the site for introducing the graft to the coronary artery 110.

In order to deliver component 10 and graft conduit 104 to the operative site during a percutaneous procedure, it is often preferable to install a longitudinal member, such as wire 98. Wire 98 passes out of aorta 106 through catheter 120 at location 102. Wire 98 enters coronary artery 110 at location 108 and may be anchored downstream along the coronary artery by anchor device 122, such as an expandable balloon. During this operation, wire 98 preferably remains within the pericardial membrane 124, as described in Berg et al. U.S. Pat. No. 6,475,222, incorporated by reference above. Apparatus and methods for deploying a longitudinal member from the aorta directly into the coronary artery is disclosed in U.S. Pat. No. 6,120,432 and incorporated by reference above.

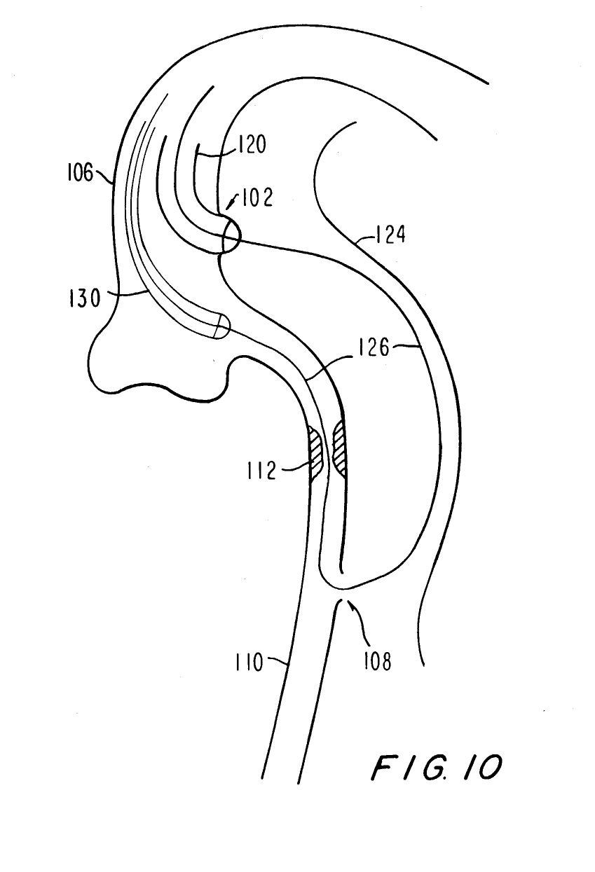

FIG. 10 illustrates an alternative procedure and apparatus in which a longitudinal member, such as wire 126, extends from aorta 106 and enters coronary artery 110 at location 108. In contrast to wire 98, above, wire 126 then passes upstream, through the occlusion 112 and into catheter 130. Both ends of wire 126 may pass outside of the patient in order to manipulate the wire 126. Under certain circumstances it may be possible or preferable to install wire 126 in this manner, particularly when occlusion 112 is not a complete blockage of the coronary artery 110. For example, Goldsteen et al. U.S. Pat. No. 5,976,178, incorporated by reference above, discloses first and second longitudinal members deployed intraluminally along and through the circulatory system. The first longitudinal member is deployed out of the aorta from a catheter (such as catheter 120) at location 102 and into the space defined by the pericardial membrane 124. The second longitudinal member is similarly deployed along the coronary artery, passing the occlusion. Subsequently, the second longitudinal member is passed from inside the lumen of coronary artery 110 to the outside thereof at location 108. The first and second longitudinal members are then interengaged, such that withdrawing the second longitudinal member pulls as much additional length of the first longitudinal member into the patient. When the second longitudinal member has been completely removed from the patient, then there is one continuous wire, i.e., the first longitudinal member, such as wire 126 in FIG. 10. Wire 126 extends from outside the patient, along and through the circulatory system, and out of aorta 106 at location 102. Wire 126 continues into the coronary artery 110 at location 108, along and through the circulatory system, to outside the patient.

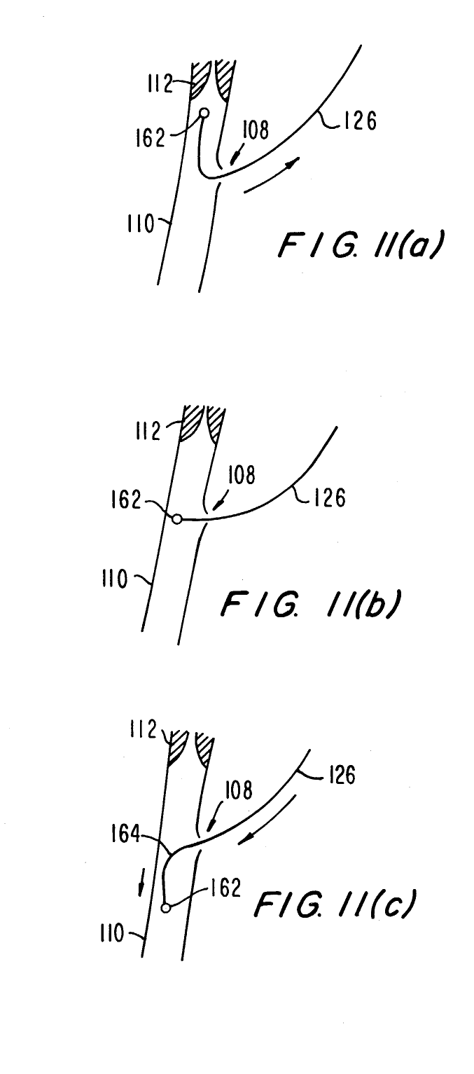

FIGS. 11(a)-11(c) illustrate an alternative apparatus and procedure for positioning a wire within the coronary artery 110 downstream of the occlusion 112. According to the alternative embodiment illustrated in FIG. 11(a), wire 126 is provided with an atraumatic tip 162, which may be a round beaded portion that is attached to an end of wire 126.

As FIG. 11(a) illustrates, wire 126 is retracted from the coronary artery 110 until the end portion having the atraumatic tip 162 is withdrawn to a location adjacent the opening in the coronary artery 110 at location 108 (FIG. 11(b)). The atraumatic tip 162 may be sized sufficiently large to prevent the wire 126 from being completely withdrawn from the coronary artery 110. Moreover, the atraumatic tip may be radiopaque in order to assist the physician during the procedure. At FIG. 11(c), wire 126 is re-advanced into the coronary artery 110. The atraumatic tip 162 prevents the end of the wire 126 from tearing the interior of the coronary artery 110 and assumes a downstream facing orientation. It is contemplated that the end portion 164 of wire 126 may be fabricated from a shape memory alloy. Consequently, when wire 126 is withdrawn to the configuration of FIG. 11(b), the end portion 164 may automatically be restored to the “L”-shaped configuration of FIG. 11(c).

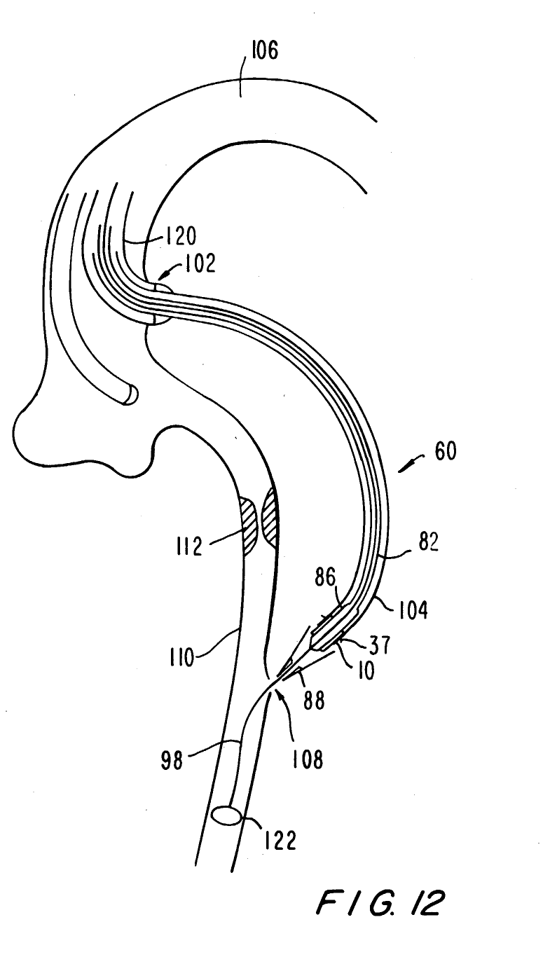

As illustrated in FIG. 12, wire 98 (or wire 126) extends from location 102 outside the aorta 106 and returns inside the circulatory system at location 108 in coronary artery 110. The graft delivery assembly 60, illustrated in greater detail in FIG. 7, above, defines a low profile for passage intraluminally within the patient. Graft delivery assembly 60 is deployed over structure 98, and may be introduced into the patient remotely and passed along and through the lumens of the circulatory system to location 102. Preferably, delivery assembly 60 is deployed from within catheter 120 into the space defined within the pericardial membrane 124. Outer sheath 92 is depicted in FIGS. 7-8 surrounding graft conduit 104. However, as illustrated in FIG. 12, it will be preferable under certain circumstances to deploy graft delivery assembly without sheath 92.

Graft delivery assembly 60 may be advanced over structure 98 to location 108. Introduction cone 88 gradually dilates the opening in the coronary artery 110 to sufficient diameter to permit the insertion of component 10, having the initial configuration of FIG. 2, and graft conduit 104.

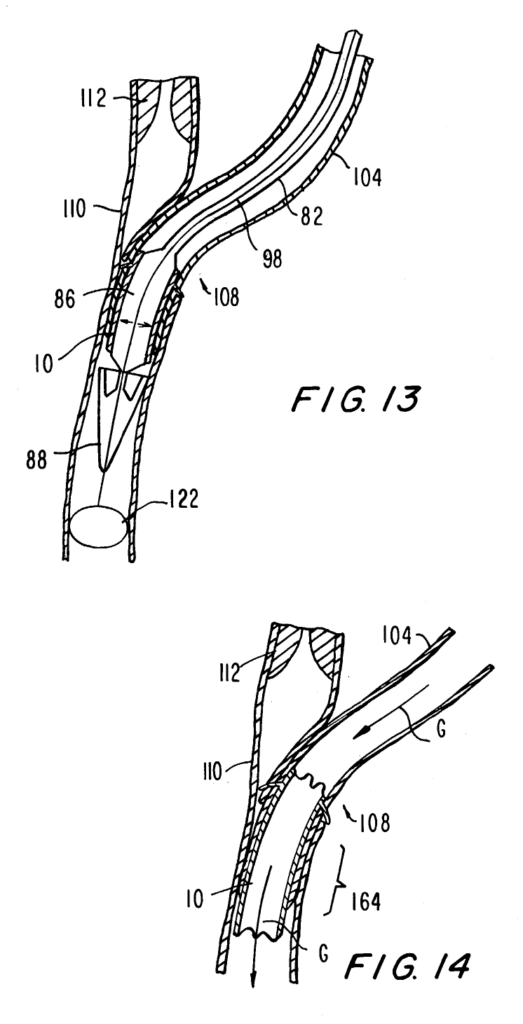

As FIG. 13 illustrates, introduction cone 88, along with component 10 and graft conduit 104 are inserted into the coronary artery 110, and are guided downstream within the lumen of the coronary artery by wire 98. After the component 10 has been inserted a predetermined amount into the opening in the coronary artery at location 108, positioning members 37 engage the wall of the coronary artery, and prevent further insertion of component 10. The engagement of members 37 with the coronary artery wall provides a tactile indication to the physician that the component 10 and graft 104 have been inserted an appropriate amount. When the physician has determined that the graft conduit 104 and component 10 are properly positioned within the coronary artery 110, the cone 88 is advanced distally and the balloon structure 86 is expanded by the introduction of compressed air, saline or other fluid through catheter 82. As balloon structure 86 expands, component 10 radially expands from the initial configuration, similar to that illustrated in FIG. 2, to a deployed configuration, having a larger diameter while maintaining a substantially constant length. As component 10 is expanded, the distal end portion 114 of graft conduit 104 is likewise expanded to conform to the inner lumen of the coronary artery 110.

Component 10 is configured to radially expand without being restrained by introduction cone 88 (See, FIGS. 7-8). In alternative embodiments, for example, the sleeve 90 and component 10 may be movable with respect to one another, rather than fixed, and oriented such that, during radial expansion, component 10 acts as a cam to urge cone 88 distally to the position shown in FIG. 13. Alternatively, cone 88 may be fabricated from frangible components which separate upon undergoing radial stresses from an expanding component 10 (not shown). As yet another alternative, lumen 96 of cone 88 may be configured with a recess to receive a bead or other projection on wire 98. Delivery assembly 60 may be advanced in the coronary artery 110 slightly downstream of the desired component placement until the bead on wire 98 is engaged with the recess in cone 88. Cone 88 is consequently fixed, and the component 10 and graft conduit 104 are withdrawn proximally to clear the component 10 of cone 88 and permit radial expansion of component 10.

FIG. 14 illustrates component 10 in a deployed configuration. Component 10 is preferably fabricated with plastically deformable materials to expand to the deployed configuration under the expansive force of the balloon structure 86 and remain in the deployed configuration after balloon structure 86 is deflated and removed. Consequently, the graft conduit 104 is secured in position with respect to the coronary artery 110. Moreover, a hemodynamic seal is established circumferentially around the distal portion 114 of the graft conduit 104 in at least the region denoted 164 in the FIG. The annular seal is formed due to the compression exerted on graft conduit 104 between component 10 and the wall of coronary artery 110. The artery wall supports and secures the graft in place. This is beneficial when a venous graft is used. Since veins are typically under a lower flow pressure than an arterial vessel, connecting the graft to the aorta 106 may place additional stress on the vein. In the subject invention, however, the vein graft 104 is reinforced by the coronary artery 110 in the region 164, which may improve patency of the graft. Moreover, it may not be necessary to block the native flow in the coronary artery 110 upstream of the distal anastomosis. As shown in FIG. 14, the insertion of the graft 104 may cause the coronary artery 110 to seal itself and obviate the need for a separate plug. FIG. 14 also illustrates that component 10 may be radially expanded within the lumen of the coronary artery 110 such that component 10 articulates to conform to the curvature of the graft conduit 104.

Once the physician has determined that component 10 is deployed, balloon structure 86 is allowed to compress and is removed from the operative site. Likewise anchor member 122 is deflated and removed with cone 88 when wire 98 is withdrawn. As indicated in the FIG. (arrows G), the flow transition from the graft conduit 104 to the coronary artery 110 is gradual and nearly parallel. The resulting blood flow thus avoids the problems of competitive upstream flow and turbulent flow that may result from the conventional anastomosis procedure illustrated in FIG. 1, above.

The proximal anastomosis, i.e., the joining of the graft conduit 104 with the wall of the aorta 106 at location 102 may be performed as disclosed in U.S. Pat. Nos. 5,976,178 and 6,152,937, which are both incorporated by reference above.

Alternatively, component 10 and graft conduit 104 may be installed in the coronary artery or other tubular body conduit by surgical delivery into the patient via an access opening T, such as an incision or a small cannula, as illustrated in FIG. 15. The methods and apparatus described above with respect to FIGS. 9-14 are applicable to surgical procedures, with the differences noted below.

The installation of wire 98 is achieved by accessing the coronary artery 110 and directly inserting wire 98 in the coronary artery 110 at the desired location downstream from the occlusion 112. The end portion of the wire 98 extends further downstream from the entry location 108 and is anchored at that location by an anchor device such as balloon anchor 122.

The graft assembly 80 (see, FIGS. 6(a)-6(b)), including component 10 and the tubular graft conduit 104 are delivered to location 108 by a delivery device 260 substantially similar to device 60 disclosed above with respect to FIGS. 7-8. More particularly, surgical delivery device 260 is shorter than those used for intraluminal delivery because the surgical apparatus is not passed from outside the patient's body and along and through the circulatory system to the graft site. As shown in FIG. 15, balloon catheter 282 is substantially similar to the balloon catheter described above with respect to FIGS. 7-8. Catheter 282 includes body portion 284 and expandable delivery structure, such as balloon structure 286. Balloon structure 286 is provided with an expansion medium, such as air or saline from supply 287, in order to expand the balloon structure 286. Introduction cone 288 has a gradual taper to gradually dilate the aperture in the coronary artery 110 at location 108. Outer sheath 272 surrounds the graft conduit 104 and component 10 during the delivery process. Sheath 272 may be provided with a flange 273 to facilitate manipulation thereof with respect to the graft 104. Balloon catheter 282 is provided with a lumen to advance the component 10 and graft conduit 104 over wire 98 and into the coronary artery 110 at location 108. Component 10 is deployed substantially as shown in FIG. 13 by expanding the balloon structure 286. The catheter 282 and outer sheath 272 are subsequently removed from the operative location, as are the introduction cone 288, wire 98 and wire anchor 122. The distal anastomosis is substantially complete. The proximal anastomosis at location 102 is subsequently performed as described in U.S. Pat. No. 6,152,937, incorporated by reference above.

The coronary artery bypass procedure may also be performed by severing one of the patient's internal mammary arteries (IMA), and reconnecting the portion of the IMA which comes from the aorta to the blocked or constricted coronary artery downstream from the blockage or constriction. Thus, the re-routed IMA supplies the blood flow needed in the downstream portion of the coronary artery. Since the IMA serves as the arterial blood source, only a single end portion of the vessel is free, in contrast with procedures which incorporate a graft having two free ends. It is contemplated that the distal anastomosis using an IMA be performed using one of several procedures in accordance with the subject invention.

One alternative embodiment is a modification to the intraluminal procedure described above with respect to FIGS. 9-14. In order to install a longitudinal structure, such as wire 98 in FIG. 14, from the end portion 182 of the IMA 180 to the coronary artery 110, the physician must sever the IMA 180, position the severed end portion 182 adjacent the coronary artery 110 at location 108, and finally deploy the longitudinal structure 98 from the IMA 180. The procedure described in U.S. patent application Ser. No. 08/869,808, incorporated by reference above, would be useful in installing longitudinal member 98 (with particular reference to FIGS. 3-7).

Connector 10 is attached to the end portion 182 of the IMA 180. Connector 10 may be introduced surgically by a small incision in the patient and positioned at the end portion 182. Alternatively, component 10 may be introduced intraluminally through the patient's circulatory system to the end portion 182. Sutures may be applied to secure component 10 to the IMA 180 by surgical access. Alternatively, connector 10 may be provided with engagement members, such as members 36, to provide attachment without sutures (See, FIGS. 2 and 6(b)).

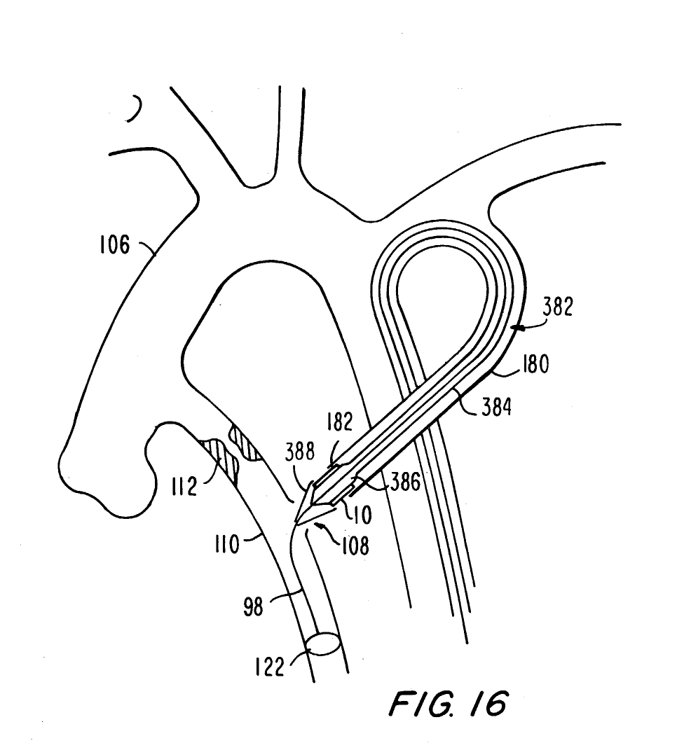

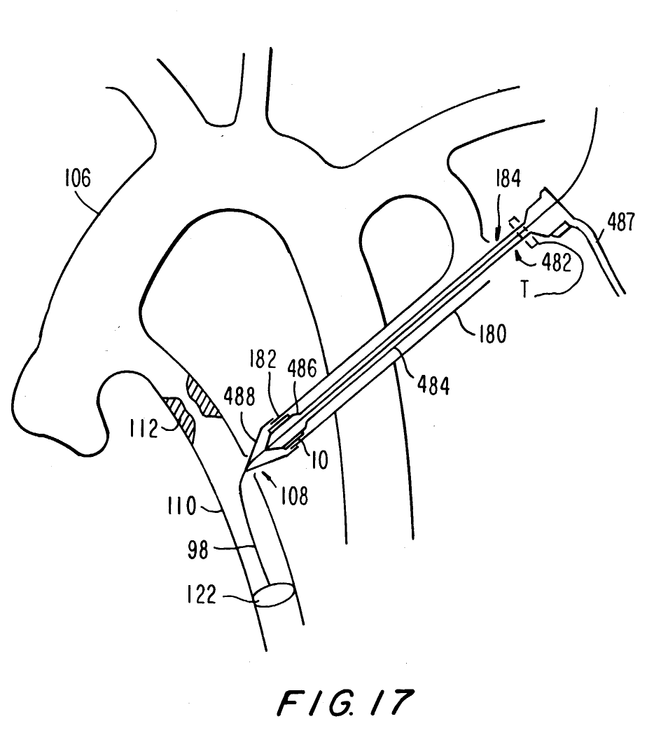

Balloon catheter 382, including balloon structure 386 and body portion 384 are introduced intraluminally over wire 98 to the severed end portion 182 of the IMA, as illustrated in FIG. 16.

Introduction cone 388 is positioned over wire 98 at the distal end portion 182 of the IMA. Cone 388 may be introduced surgically by a small incision in the patient and positioned at the end portion 182. Alternatively, cone 388 may be introduced intraluminally through the patient's circulatory system to the end portion 182 simultaneously with balloon catheter 382. Balloon structure 386 engages the inner surface of component 10. (This may be achieved by frictional engagement, such as by advancing balloon structure 386 within component 10 and slightly inflating balloon structure 386). Further advancement of the balloon catheter 386 advances component 10 and the IMA 180 therewith. Component 10 is installed in the lumen of the coronary artery 110, substantially as described above with respect to FIGS. 13-14.

Another alternative embodiment is illustrated in FIG. 17, which is similar to the apparatus and methods described above with respect to FIG. 14. Connector 10 is attached to the end portion 182 of the IMA 180. In order to surgically install the IMA 180 in the coronary artery 110, an arteriotomy 184 is made remote from the severed end portion 182. The delivery apparatus, including balloon catheter 482, would be inserted into the patient via an access opening T, such as an incision or a small cannula, and into arteriotomy 184 and along and through the IMA 180, to the end portion 182 adjacent component 10. Installation of the end portion of the IMA proceeds substantially as described above. After installation is completed, catheter 482, introduction cone 488, and wire 98 are withdrawn. Sutures or other closing means are applied to the IMA at the arteriotomy 184 to complete the procedure.

It will be understood that the foregoing is only illustrative of the principles of the invention, and that still other modifications can be made by those skilled in the art without departing from the scope and spirit of the invention. For example, the various materials and dimensions mentioned herein are only examples, and other materials and dimensions can be used if desired.

The invention claimed is

1. Apparatus for securing an axial end portion of a new length of tubing in a lumen of a patient's existing tubular body organ structure so that the lumen of the new length of tubing is partially coaxially disposed within the lumen of the existing tubular body organ structure and communicates with the lumen of the existing tubular body organ structure via an aperture in a side wall thereof to permit fluid flow between the lumens, comprising:
an anchor device, having a body portion and an attachment member, the attachment member deflected radially outwardly from the body portion and adapted for penetration of both the end portion of the new length of tubing and the existing tubular body organ structure, the body portion defining a substantially constant axial length and a cross-section expandable between a first diameter sized for insertion into the aperture in the side wall of the body conduit and a second diameter sized to secure the end portion of the new length of tubing coaxially between the anchor device and the lumen of the existing tubular structure, wherein the body portion has a plurality of axially extending members having axial lengths that remain substantially constant during radial expansion; and
a delivery device configured for coaxial insertion into the existing tubular structure and configured to expand the anchor device to the second diameter, wherein the delivery device further comprises a tapered structure positioned distal to the anchor device to facilitate introduction of the anchor device into the existing tubular structure, wherein the tapered structure partially surrounds the body portion during said introduction of the body portion into the existing tubular structure.

2. Apparatus defined in claim 1, wherein the tapered structure is relatively movable with respect to the body portion into the existing tubular structure to a position spaced apart from the body portion during the expansion thereof between the first and second diameters, and wherein the tapered structure is configured for withdrawal within and through the body portion following the expansion.

3. Apparatus defined in claim 1, wherein the anchor device has a plurality of axial members and a plurality of circumferential members connected to the axial members, each circumferential member having a first configuration and a second configuration relatively expanded with respect to the first configuration.

4. Apparatus defined in claim 3, wherein the circumferential members are deformable between the first and the second configuration.

5. Apparatus defined in claim 4, wherein the anchor device is configured such that the deformation of the circumferential members produces relative radial displacement of adjacent axial members with respect to one another.

6. Apparatus defined in claim 3, wherein the first configuration of the circumferential members is substantially serpentine.

7. Apparatus defined in claim 1, wherein the anchor device defines an aperture configured to receive a suture for securing the end portion of the new length of tubing to the anchor device.

8. Apparatus defined in claim 1, wherein the anchor device defines a longitudinal axis movable between a substantially straight configuration and a curvilinear configuration such that the cross-section remains substantially unchanged in said configurations.

9. Apparatus defined in claim 8, wherein the anchor device comprises a plurality of axial members, each having an expansion link to permit elongation of the respective axial member.

10. Apparatus defined in claim 9, wherein the expansion link is configured for independent expansion with respect to an adjacent expansion link.

11. Apparatus defined in claim 9, wherein the expansion link is plastically deformable between a compressed and an expanded configuration.

12. Apparatus defined in claim 11, wherein the expansion link has a substantially “V”-shaped configuration.

13. Apparatus defined in claim 12, wherein the expansion link extends radially outward and is configured to receive a suture for securing the graft conduit to the anchor device.

14. Apparatus defined in claim 1, wherein the anchor device comprises a positioning member extending radially outward from said anchor device and configured to engage the side wall of the existing tubular structure.

15. Apparatus defined in claim 1, wherein the anchor device comprises a positioning member extending radially outward from said anchor device and positioned at a predetermined location along the axial length thereof and configured to engage the side wall of the existing tubular structure when the anchor device has been inserted a predetermined distance into the existing tubular structure.

16. Apparatus defined in claim 1, wherein the anchor device comprises a positioning member extending radially outward from said anchor device and positioned at a predetermined location along the axial length thereof and configured to provide a tactile indication to a user upon engagement with the side wall of the existing tubular structure when the anchor device has been inserted a predetermined distance into the existing tubular structure.

17. Apparatus defined in claim 8, wherein the anchor device comprises a plurality of axial members configured for relative radial movement and differential axial elongation with respect to adjacent axial members.

18. Apparatus defined in claim 17, wherein the axial members are interconnected by circumferential members deformable between a compressed and an expanded configuration.

19. Apparatus defined in claim 18, wherein each of the axial members has an expansion link configured to allow elongation of the axial member.

20. Apparatus defined in claim 1, further comprising:
a delivery device configured for coaxial insertion into the existing tubular structure and configured to expand the anchor device to the second diameter.

21. Apparatus defined in claim 20, wherein the delivery device further comprises a tapered structure positioned distal to the anchor device to facilitate introduction of the anchor device into the existing tubular structure.

22. Apparatus defined in claim 20, wherein the delivery device defines an axial bore, and the apparatus further comprises:
an elongated guide structure slidably received in the axial bore and configured for insertion into the existing tubular structure.

23. Apparatus defined in claim 1, wherein the attachment member is plastically deformable to the position deflected radially outwardly from the body portion.

24. Apparatus defined in claim 1, wherein the attachment member is configured to receive a suture for attaching the new length of tubing to the anchor device.

25. Apparatus defined in claim 1, wherein the body portion has an articulation cell that allows the body portion to conform to a curve of the existing tubular body organ structure.
